(19) United States
(12) Patent Application Publication
(10) Pub. No.: US 2009/0274749 A1
(43) Pub. Date: Nov. 5, 2009

(54) Topical Application for Minimizing Melanin Production

(76) Inventor: Benjamin Johnson, Evergreen, CO (US)

Correspondence Address: GLENN L. WEBB;GLENN L. WEBB P.C., P.O BOX 3788, DURANGO, CO, 81302, US

(21) Appl. No.: 12/113,115

(22) Filed: Apr. 30, 2008

(51) Int. Cl. A61K 9/127; A61K 8/14; A61Q 19/00

(52) U.S. Cl. 424/450; 424/62

(57) ABSTRACT

Tyrosinase inhibitors are incorporated with liposomes in therapeutic compositions for topical application to hyperpigmentation. In particular, combinations of tyrosinase inhibitors are combined with liposomes to increase the penetration of the tyrosinase inhibitors into the deep epidermis skin layers and to provide hydration of the skin during treatment.

### FIELD OF THE INVENTION

This invention relates to the field of controlling the production of melanin through tyrosinase inhibitors.

### BACKGROUND OF THE INVENTION

Many individuals are bothered by the color and tone of their skin, particularly for age-related dark spots, skin pigmentation, freckles and to lighten skin tones. Skin color differentiation is due primarily to the nature and quantity of the natural skin pigment, referred to as melanin. The darkening of the pigmentation of skin is largely due to the production of melanin from melonocytes in the epidermis skin layer. Melonocytes are normally located in the basal layer of the epidermis and contain unique organelles called melanosomes. The melanosomes cluster and redistribute within the melonocytes based on external cues such as ultraviolet radiation as well as stress, acne, hormones, inflammatory processes or free radical damage. Melanin is biosynthesized in the melanosome of a melanocyte from the amino acid tyrosine by an enzyme called tyrosinase. The tyrosinase transforms tyrosine into 3,4-dihydroxyphenylalanine (DOPA), then to DOPAquinone and then to 2-carboxy-2,3-dihyrodindole-5,6-quinone (DOPAchrome). DOPAchrome is then converted by other enzymes into more highly oxidized materials into melanin (eumelanin, phaelomelanin) supplying the pigmentation for the skin.

Melanin thus provides the pigmentation for the color of the skin but it is also an important factor in protecting the skin from harmful effects of ultraviolet rays. Ultraviolet rays activate the melanosome in the melonocytes causing increased production of melanin, thus darkening the skin. This only occurs from the presence of the tyrosinase enzyme that causes the oxidation process creating melanin.

Problems associated with the production of melanin result from excessive amounts of melanin causing darkening of the skin, freckles, dark skin spots, and the non-uniform distribution of melanin causes chloasma and ephelis. Also, melanoma, a skin cancer tumor, can also result from the activation of melanosome in a malignant melanocyte. Tyrosinase activity from ultraviolet exposure can result in DNA damage to the melanocyte increasing the risk of melanoma.

One technique for lessening the effects of skin pigmentation, whitening the skin and protecting from melanoma are to use tyrosinase inhibitors. Tyrosinase inhibitor agents inhibit the enzymic action of tyrosinase in the melanocyte prior to, during or after ultraviolet radiation exposure. There are a number of naturally occurring tyrosinase inhibitor agents as well as chemically prepared agents that have been used in cosmetic lightening applications.

These agents have differing degrees of success depending on many factors including differing mechanisms for inhibiting the tyrosinase enzymic activity. Also, most of the agents are relatively unstable. Many of these agents tend to irritate the skin as well. Also, the efficacy of the agents depend on their ability to penetrate the lower levels of the epidermis and penetrate the melanocytes in order to inhibit the enzymic activity of the tyrosinase.

### SUMMARY OF THE INVENTION

The present invention provides a topical skin care product that will treat hyperpigmentation. It is also believed that it may reduce the risk of melanoma. The skin care product of the present invention treats these problems by reducing the enzymic activity in the melanocytes in the deep epidermis skin layer. This reduces the production of melanin which reduces the hyperpigmentation.

In a preferred embodiment, the skin care product of the present invention uses a combination of tyrosinase inhibitor agents to more effectively penetrate the epidermis layer and to act upon the melanocytes to inhibit the enzymic activity of the tyrosinase. This combination of tyrosinase inhibitor agents provide a more effective product. Each of the tyrosinase inhibitor agents have a differing mechanism for inhibiting the tyrosinase enzymic activity in the meloncytes.

In another preferred embodiment the skin care product of the present invention uses one or more tyrosinase inhibitor agents contained within a liposome compound. This provides a vehicle for the tyrosinase inhibitor agents to more effectively penetrate the epidermis layer to the basal epidermal layer where the melanocytes are located. The liposomal compound also renders the tyrosinase inhibitor agents to be more stable, particularly when mixed with other cosmetic ingredients. The liposomal compound of a preferred embodiment of the present invention also hydrates the skin during application and reduces irritation to the skin.

The skin care product of a preferred embodiment uses a combination of tyrosinase inhibitor agents. In the preferred embodiment, the product is formed from combinations selected from the group of tyrosinase inhibitor agents that each have differing mechanisms for affecting the enzymic activity in the melonocytes.

The skin care product of the present invention is able to use a combination of differing tyrosinase inhibitor agents contained within a liposome compound to provide a skin care product that when topically applied is effective at penetrating the epidermis layer to reduce the enzymic activity of the melanosomes in creating melanin without irritating the skin and reducing harmful side effects. It is effective in treating hyperpigmentation, in reducing the risk of melanoma and in treating damage incurred before, during and after overexposure to ultraviolet radiation.

These and other features of the present invention will be evident from the ensuing detailed description of preferred embodiments and from the claims.

### DETAILED DESCRIPTION OF PREFERRED EMBODIMENTS

The present invention provides products and methods for reducing the production of melanin to lighten the skin and to reduce the risk of melanoma. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the claimed invention. It is to be expressly understood that other products and methods are contemplated for use with the present invention as well.

In accordance with the present invention, combinations of tyrosinase inhibitors are incorporated with liposomes in therapeutic compositions for topical application to prevent or alleviate the conditions and symptoms of skin disorders such as hyperpigmentation and other skin disorders related to the production of melanin are described as follows. In particular, combinations of tyrosinase inhibitors are combined with liposomes to decrease the production of melanin, to increase the penetration of those tyrosinase inhibitors into the skin, to provide hydration of the skin during treatment, and other embodiments and uses.

### Tyrosinase Inhibitors

There are numerous tyrosinase inhibitors currently available from natural sources as well as chemically prepared sources. Essentially, tyrosinase inhibitors are agents that penetrate into the melanocytes deep in the epidermis layer to prevent or minimize the production of tyrosinase. Melanin is not able to be produced in the melanosomes in the absence of tyrosinase. Thus, hyperpigmentation is reduced as well as possibly reducing the occurrence of melanoma.

Tyrosinase inhibitors tend to be relatively unstable, particularly when used in cosmetic applications. Many are unable to effectively penetrate sufficiently deep into the epidermis layer to reach the melanocytes. Most have differing life spans, thus it is difficult to create an effective treatment plan. There are also issues with toxicity and sensitivity from many of these agents.

Examples of tyrosinase inhibitor agents include, but are not limited to, kojic acid and its derivatives, arbutin and its derivatives, licorice extract and its derivatives, ascorbic acid and its derivatives, hydroquinone and its derivatives, lipoic acids, thiopronions, glutathione, pantetheines, flavonols, pyrones, scutellaria extract, mulberry extract, burner root extract, paper mulberry bark, rumex occidentalis, licorice extract, alpha arbutin, GABA, Bearberry, Watercress Extract, Tetrahydrocurcuminoids, Sepiwhite (a proprietary product of Seppic, a division of Air Liquide group), melanostatin, thiotaine, tyrostatase, asafetida extract, lemon peel extract, wheat germ extract, citris unshiu peel extract, creatine as well as many others.

The preferred embodiment of the present invention incorporates combinations of selected tyrosinase inhibitor agents to create an effective product that is stable, able to be used in cosmetic preparations and can deeply penetrate the epidermal layer to act on melonocytes to inhibit the enzymic activity of tyrosinase, thus reducing the production of melanin. In the preferred embodiment, the product is formed from combinations selected from a group of tyrosinase inhibitors that have differing mechanisms for affecting the tyrosinase enzymic activity in melanocytes. It is to be understood that other tyrosinase inhibitor agents may be used as well.

In the preferred embodiment, a combination of at least seven tyrosinase inhibitors are combined where each of the selected tyrosinase inhibitors have a differing mechanism for affecting the tyrosinase enzymic activity. This provides much greater efficacy in the reduction of melanin.

### Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Preparation of Exemplary Therapeutic Compositions

To prepare a typical aqueous solution, combinations of the selected tyrosinase inhibitor agents are dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, combinations of the selected tyrosinase inhibitor agents are dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved.

typical cream or lotion containing combinations of the selected tyrosinase inhibitor agents in ethanol, acetone, propylene glycol or other solvent. The solution thus prepared is then admixed with commonly available oil-in-water emulsions. BHT or sodium metabisulfite may be added to such emulsions to stabilize the combinations of the selected tyrosinase inhibitor agents. Liposomes or liposome substitutes are added to this solution with mixing until a uniform consistency is achieved.

A typical gel composition is formulated by first dissolving combinations of the selected tyrosinase inhibitor agents in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

Therapeutic Uses

The present invention may in various embodiments be used to increase the efficacy of the use of tyrosinase inhibitor agents for reducing the production of melanin to treat hyperpigmentation and to reduce the risk of melanoma. A preferred embodiment of the present invention increases the efficacy of a topical skin care product by increasing the penetration of combinations of the selected tyrosinase inhibitor agents into the skin. Compositions containing combinations of the selected tyrosinase inhibitor agents are coated or mixed with liposomal materials as described above. The liposomal combinations of the selected tyrosinase inhibitor agents compound has been shown to increase the penetration of combinations of the selected tyrosinase inhibitor agents which increases the efficacy of the combinations of the selected tyrosinase inhibitor agents.

The preferred embodiment of the liposomal tyrosinase inhibitors allow deeper penetration of this beneficial activity that has not been possible in prior topical compositions.

The effect of combinations of the selected tyrosinase inhibitor agents to treat hyperpigmentation occurs at the deep epidermis level where the melanocytes occur, so without adequate penetration, they have limited function in the skin. Previous tyrosinase inhibitors often irritate the skin. The liposomal tyrosinase inhibitors compound has been shown to greatly increase the penetration of the tyrosinase inhibitors which increases the efficacy of the product.

Another preferred embodiment of the present invention increased the hydration of the skin from using tyrosinase inhibitors. Compositions containing tyrosinase inhibitors are coated or mixed with liposomal materials as described above. Liposomes have a natural affinity for water which assists in increasing the moisture in the skin during topical application of the liposomal tyrosinase inhibitors composition.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

1. A method for treating hyperpigmentation with a topical skin care application, wherein the method comprises:
providing a topical skin care application;
providing a combination of seven or more tyrosinase inhibitors in said skin care application to increase the stability of the skin care application and to increase the efficacy of the skin care application;
providing said combination of seven or more tyrosinase inhibitors within a liposome compound to increase the penetration of said combination of two or more tyrosinase inhibitors into the deep epidermis skin layer; and
applying said skin care application to the skin to treat the hyperpigmentation by reducing the tyrosinase enzymic activity in the melanocytes.

2. The method of claim 1 wherein said step of providing a combination of seven or more tyrosinase inhibitors further includes:
said combination of seven or more tyrosinase inhibitors are selected from the group of tyrosinase inhibitors where each of the inhibitors affect the tyrosinase enzymic activity in the melanocytes in a different manner that the other inhibitors.

3. The method of claim 1 wherein said step of providing said combination seven or more tyrosinase inhibitors within a liposome compound further includes:
providing said combination of tyrosinase inhibitors within a liposome compound to hydrate the skin during the application.

4. The method of claim 1 wherein said step of providing said combination seven or more tyrosinase inhibitors within a liposome compound further includes:
providing said combination of tyrosinase inhibitors within a liposome compound to reduce irritation to the skin during the application.

5. The method of claim 1 wherein said method further comprises:
treating the skin with said skin care application before, during and after overexposure to ultraviolet radiation to reduce the risk of the occurrence of melanoma.

6. The method of claim 1 wherein said method further comprises:
treating the skin with said skin care application to lighten the color and tone of the skin.

7. The method of claim 1 wherein said skin care application further includes:
an aqueous solution.

8. The method of claim 1 wherein said skin care application further includes:
a non-aqueous solution.

9. The method of claim 1 wherein said skin care application further includes:
a gel solution.

10. The method of claim 1 wherein said skin care application further includes:
a lotion solution.

11. A skin care product for treatment of hyperpigmentation, said skin care product comprising:
a liposome compound; and
seven or more tyrosinase inhibitor agents contained in said liposome compound.

12. The skin care product of claim 11 wherein said skin care product includes:
said combination of seven or more tyrosinase inhibitors are selected from a group of tyrosinase inhibitors where each of the inhibitors affect the tyrosinase enzymic activity in the melanocytes in a different manner that the other inhibitors.

13. The skin care product of claim 11 wherein said skin care product further includes:
an aqueous solution.

14. The skin care product of claim 11 wherein said skin care product further includes:
a non-aqueous solution.

15. The skin care product of claim 11 wherein said skin care product further includes:
a gel solution.

16. The skin care product of claim 11 wherein said skin care product further includes:
a lotion solution.
